(19) United States
(12) Patent Application Publication
(10) Pub. No.: US 2013/0344605 A1
(43) Pub. Date: Dec. 26, 2013

(54) METHOD AND ARRANGEMENT FOR PARTIAL LABELING AND SUBSEQUENT QUANTIFICATION OF CELLS OF CELL SUSPENSION

(71) Applicants: Oliver HAYDEN, Herzogenaurach (DE); Michael Johannes HELOU, Regensburg (DE); Lukas RICHTER, Erlangen (DE)

(72) Inventors: Oliver HAYDEN, Herzogenaurach (DE); Michael Johannes HELOU, Regensburg (DE); Lukas RICHTER, Erlangen (DE)

(73) Assignee: SIEMENS AKTIENGESELLSCHAFT, Munich (DE)

(21) Appl. No.: 13/924,171

(22) Filed: Jun. 21, 2013

(30) Foreign Application Priority Data
Jun. 21, 2012 (DE) 102012210457.7

(51) Int. Cl. G01N 33/58

(52) U.S. Cl. CPC G01N 33/585
USPC 436/63; 422/502; 422/73

(57) ABSTRACT

Cells of a cell suspension are labeled by providing a microfluidic chamber having superparamagnetic labeling particles that are concentrated exactly at one inner surface of the chamber and charging the cell suspension into the chamber.

### CROSS REFERENCE TO RELATED APPLICATIONS

This application is based on and hereby claims priority to German Application No. 10 201 2 210 457.7 filed on Jun. 21, 2012, the contents of which are hereby incorporated by reference.

### BACKGROUND

Described below are a method and an arrangement or apparatus for the labeling of cells of a cell suspension using superparamagnetic labeling particles which are provided for this purpose, in particular with a specific antibody.

The labeling of cellular material in a cell sample by diverse labeling methods permits selection thereof from and quantification in complex cell samples. Methods are known which permit quantification of cellular material on the basis of counting single cells. Diverse continuous-flow cytometric methods permit the quantification of cells using fluorescence-based or magnetic selective labeling. However, these methods disadvantageously require a labor-intensive workup of the sample that is to be measured. Workup of the sample can require working steps such as, for example, centrifugation, filtration, sedimentation and many others. Frequently, such working methods are associated with a partial loss of the analyte.

There is a basic problem here with known methods in cytometry of labeling and finally counting the low number of cells present in the cell sample. Therefore, the known methods are directed toward generating an adequate signal from low amounts of labeled cells. It is disadvantageous with the known methods of continuous-flow cytometry that they are only able to handle with difficulty a cell sample that contains a high number of cells that are to be labeled, for example having a density of 10⁴ cells/μl, since, inter alia, owing to the high receptor concentration—many cells—for a sufficiently good magnetic labeling, many labeling particles are required.

### SUMMARY

Described below are a method and an arrangement for quantifying cells of a cell sample which minimizes the problem mentioned at the outset, in particular, therefore, is also able to handle cell samples having a relatively high concentration of cells that are to be detected.

In the method for labeling cells of a cell suspension, first a microfluidic chamber is provided having superparamagnetic labeling particles concentrated on substantially one inner surface of the chamber, with the labeling particles having in particular a specific antibody for binding to cells. Second, the cell suspension is charged into the chamber.

Advantageously, this achieves labeling of an accurately determinable fraction of the cells in a suspension. Only those cells of the cell suspension which flow in the region of the inner surface on which the labeling particles are concentrated are also labeled. Cells which do not flow in the region of this inner surface do not have any contact with the labeling particles and therefore are also not labeled.

In other words, therefore, the procedure means that a defined fraction of the cells is labeled, but also substantially only this defined fraction is labeled.

It is of importance in this case that in the microfluidic chamber, on account of the dimensions thereof, a substantially laminar flow prevails. In this case the various layers of the cell suspension flowing through scarcely mix with one another, and so substantially only those cells are labeled which come into the immediate vicinity of that inner surface which is occupied by the labeling particles. This is substantially a diffusion-dependent operation: only platelets which impact the labeling particles can bind to them. From this point in time they start to roll off and take up in this process still more labeling particles.

Therefore, the fraction of labeled cells can readily be determined from the thickness of the chamber perpendicular to the inner surface and the thickness of the layer of the cell suspension which is subject to labeling.

The method described here therefore permits controlled, partial and loss-free labeling of cellular material with superparamagnetic nano- or microparticles within small volumes of complex samples in a microfluidic system. In this case, advantageously, no dilution is necessary. The cellular material which is already present in large amounts need not be enriched in advance or separated from other cell types. The system presented advantageously simplifies the purification and labeling of cells in complex samples such as body fluids or body secretions.

It is advantageous here if a chamber is used that has a cavity that is substantially cuboidal, i.e., a rectangular, or square, parallelogram. Owing to the simple geometry, the fraction of labeled cells may be determined particularly readily thereby.

In addition, it is advantageous if the chamber has a thickness between 10 μm and 1000 μm, wherein the thickness describes the extension perpendicular to the inner surface on which the labeling particles are concentrated. In the range of these thicknesses, a laminar flow may be best achieved and at the same time a fraction of the cells labeled which is in the percentage or permillage range. By establishing this fraction via the thickness of the chamber, it is also possible to avoid a cell suspension needing to be diluted.

In order to provide the microfluidic chamber, the following operations are carried out:
charging the microfluidic chamber with a suspension having the labeling particles,
concentrating the labeling particles on the inner surface of the chamber by a magnet.

For this purpose, a permanent magnet may be used. Alternatively, however, it is also possible to use an electromagnet.

The charging of the cell suspension into the chamber is expediently performed in one pumping operation which simultaneously removes from the chamber the remainder of the suspension containing the labeling particles. The charging into the chamber can then be interrupted in such a manner that the cell suspension remains static in the chamber for a resting time. For example, as resting time, a time of one second or less can be chosen. The resting time increases the fraction of labeled cells in the region of the inner surface owing to the action of gravity, for example, but also permits cell diffusion in the chamber, as a result of which cells can be labeled which were not actually near the inner surface in the labeling region. Alternatively, the cell suspension can be conducted through the chamber without interruption.

In both cases, the cell suspension can be conducted to a measuring device, for example, for counting the labeled cells.

In an embodiment, outside the chamber, but in the region of the inner surface of the chamber, a coil with an iron core is arranged. The iron core serves for shaping the magnetic field which attracts the labeling particles to the inner surface. Advantageously, this means that the labeling particles lie more uniformly on the inner surface, since inhomogeneities of the magnetic field are reduced by the iron core. An accumulation of the labeling particles toward one end of the chamber is reduced in this manner. It is expedient in this case if an outer surface of the iron core that is directed toward the inner surface of the chamber has at least the dimensions of the inner surface. Ideally, the iron core is somewhat greater than the microfluidic chamber. The coil expediently generates a magnetic field which is greater, in particular substantially greater, than that of the magnet.

In a development, the iron core has indentations on the outer surface directed toward the inner surface of the chamber. These may be arranged in a regular pattern. The indentations can be, for example, parallel lines, or, for example, can follow a square line pattern. The shape of the deposition of the labeling particles can be determined thereby.

Alternatively, for use of an iron core, it is also possible to provide the inner surface itself with indentations. An accumulation of the labeling particles toward one end of the chamber is likewise thereby decreased, since the labeling particles collect within the indentations and can scarcely move between the indentations under the influence of the magnetic field.

### BRIEF DESCRIPTION OF THE DRAWINGS

These and other objects and advantages of the present invention will become more apparent and more readily appreciated from the following description of an exemplary embodiment, taken in conjunction with the accompanying drawings of which:

FIG. 1 is a schematic diagram of an apparatus for cell measurement using a microfluidic system,

FIGS. 2A-2D are schematic diagrams of a microfluidic chamber during a series of operations in preparation for cell measurement,

FIG. 3 is a schematic diagram of part of the microfluidic chamber with partially labeled cells,

FIG. 4 is a block diagram of an embodiment of the apparatus with an additional iron core,

FIGS. 5A and 5B are schematic diagrams of embodiments of the apparatus with an iron core which has indentations,

FIG. 6 is a schematic diagram of an embodiment of the apparatus with indentations in a wall of the microfluidic chamber.

### DETAILED DESCRIPTION OF THE PREFERRED EMBODIMENT

Reference will now be made in detail to the preferred embodiments of the present invention, examples of which are illustrated in the accompanying drawings, wherein like reference numerals refer to like elements throughout.

### Exemplary Embodiment 1

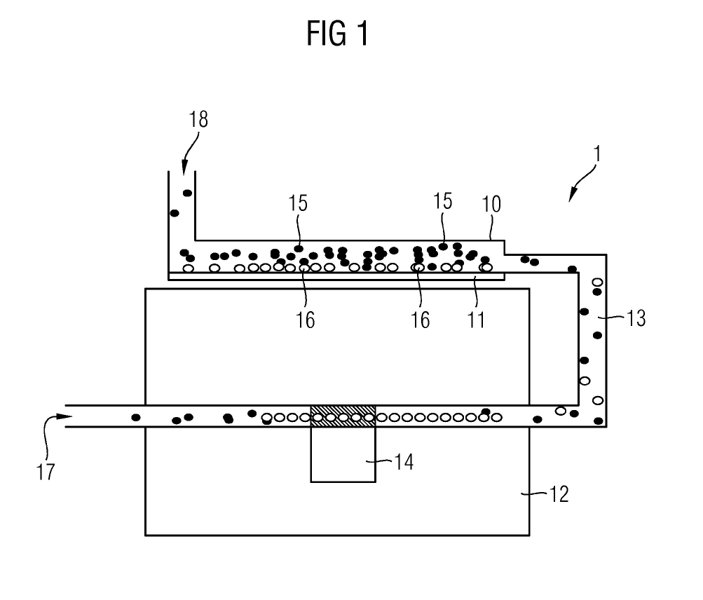

The exemplary embodiment hereinafter relates to an arrangement 1 for cell measurement according to FIG. 1, in which cells 15, 16 of a cell suspension of a very high cell density are labeled and are then fed to a counting device 14 in order to perform quantification. The arrangement 1 in this case has a permanent magnet 12 arranged on a shared support, on the surface of which permanent magnet is built up a microfluidic system. The microfluidic system includes in this case a sample feed 18 for feeding liquids into a microfluidic channel 13.

The sample feed 18 conducts the liquids into a microfluidic chamber 10, in which labeling of cells is performed. The chamber 10 in this case is implemented on the permanent magnet. On the exit side, the microfluidic system further leads to a counting device 14 in which labeled cells can be counted.

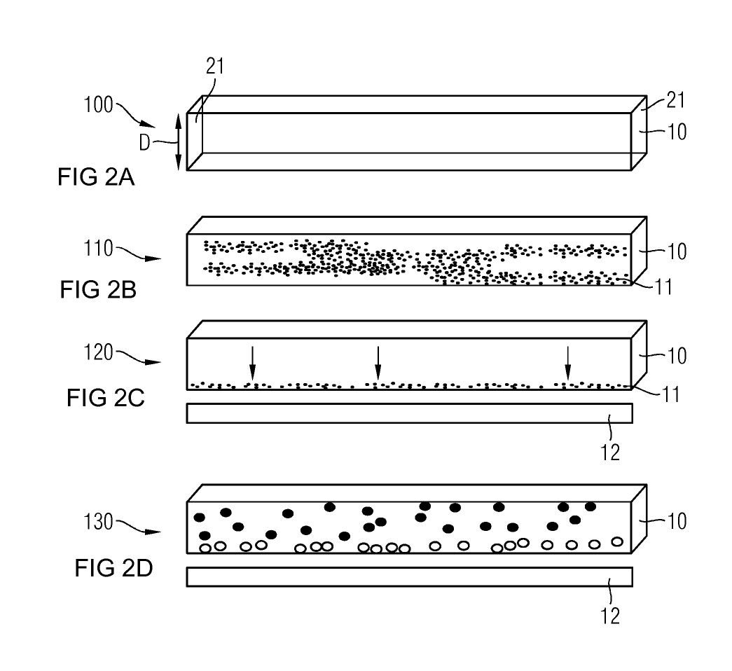

The counting device 14 is based in the present example on detection of magnetic fields, for example GMR, AMR or TMR. An excessively high concentration of labeled cells in the liquid which is passed by the counting device 14 leads in this case to saturation of the sensor and thereby to falsification of the measurement. Therefore, for the cell suspension used here in which there is a cell density of, for example, 10⁵ cells/μl, a special labeling method is used, the preparation of which is described in more detail hereinafter with reference to FIG. 2.

FIGS. 2A-2D illustrate the preparation of the microfluidic chamber 10 for cell labeling, which microfluidic chamber is prepared as illustrated in FIG. 2A. The microfluidic chamber 10 can advantageously be produced by an injection molding technique. In the present example, the thickness D of the microfluidic chamber 10 may be D=100 μm. However, other dimensions are also possible such as, for example, D=10 μm, D=50 μm, or D=980 μm. The microfluidic chamber 10, on the end faces 21 thereof, has an inlet opening and an outlet opening which are not shown in FIGS. 2A-2D. The total volume of the chamber in the present example is 1 μl, wherein the chamber is 10 mm long and 1 mm high for the previously mentioned thickness of 100 μm.

As illustrated in FIG. 2B, a suspension containing superparamagnetic labeling particles 11 is introduced into the microfluidic chamber 10. The labeling particles 11 in this case typically still do not display specific division in the suspension, but are first uniformly distributed. Furthermore, the labeling particles 11 in the present example have specific antibodies. In alternative embodiments, labeling particles 11 without antibodies can also be used.

As illustrated in FIG. 2C, then, the labeling particles 11 are attracted by the action of magnetic force, in this example via the permanent magnet 12, to the bottom of the microfluidic chamber 10.

As illustrated in FIG. 2D, the suspension—minus the labeling particles 11 retained by the permanent magnet 12—is removed from the microfluidic chamber 10. In this case the solvent of the labeling particles 11 initially charged is replaced by air before the actual analyte solution arrives. The labeling particles 11 are dried thereby. The duration of operation illustrated in FIG. 2D can be influenced by the volume of the entire system (from the inlet up to the chamber). During the operation illustrated in FIG. 2D, a cell suspension with initially unlabeled cells 15 is introduced into the microfluidic chamber 10. This occurs in this example by suction at one end 17 of the microfluidic channel 13 which leads from the microfluidic chamber 10 via the cell measuring device 14.

If the cell suspension is situated in the microfluidic chamber 10, it is allowed to stand there for a very short time, for example half a second. In this time, the unlabeled cells 15 of the cell suspension remain substantially static in the microfluidic chamber 10, and formation of layers or precipitation virtually does not take place. Those unlabeled cells 15 which come into the vicinity of the paramagnetic labeling particles 11, however, bind thereto and thus become labeled cells 16. The labeled cells 16 are thus formed only in a well-defined region close to the bottom of the microfluidic chamber 10.

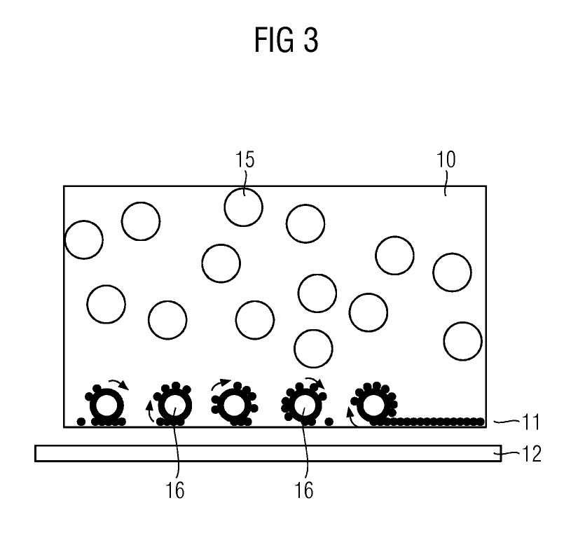

FIG. 3 shows schematically what occurs during charging of the microfluidic chamber 10 with the cell suspension. That part of the cells which is situated close to the inner surface with the labeling particles 11 rolls off on the inner surface of the chamber and in the course of this frequently receives a multiplicity of the labeling particles 11. Therefore, while the part of the cells 15 geometrically remote from the inner surface remains substantially unlabeled, the labeled cells 16 are provided with a considerable amount of labeling particles 11.

In the present example, all the unlabeled cells 15 are potentially labeled which are situated in a region of 5 μm from the bottom of the chamber 10 downwards. Since it can be assumed that virtually all cells 15 which are located in this region actually also become labeled on the passage through the chamber 10, in a simple calculation, 5% of the cells 15 become labeled cells 16. In this case, for increasing the accuracy, the diameter of the cells can also be taken into account.

Since only a comparatively small, but well defined fraction of the cells 16 is labeled, the counting device 14 can perform a cell counting without problems and the actual cell density can be estimated.

The microfluidic system can advantageously in this case be designed in such a manner that an exchange of the chamber 10 containing the labeling particles 11 is readily possible. Thus, via the choice of the labeling particles 11 or chamber geometry, the labeling can be adapted to the type of cell suspension present.

The arrangement of the microfluidic chamber 10 on the permanent magnet 12 can be problematic: Since the labeling particles 11 follow the magnetic field, they accumulate toward the end of the chamber 10, which is virtually at the center 44 of the permanent magnet 12. In order to avoid this, there are various possibilities:

### Exemplary Embodiment 2

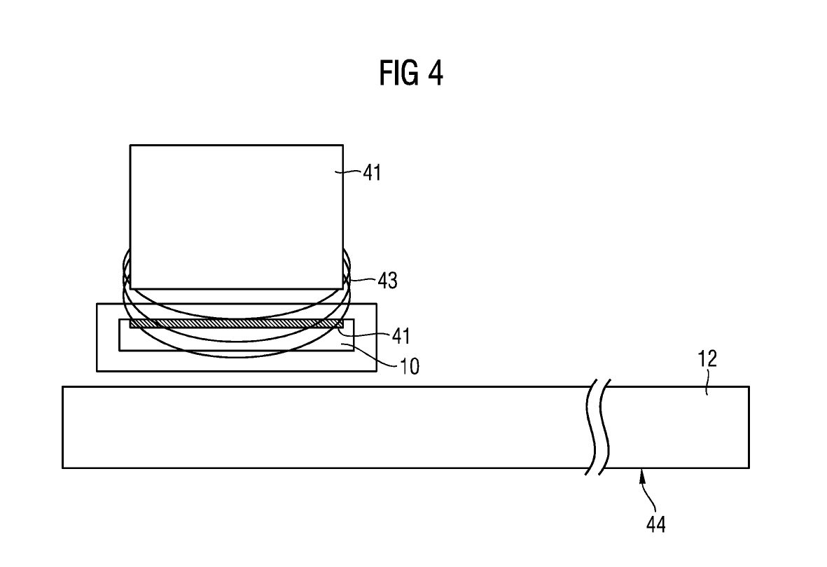

As shown in FIG. 4, a coil 41 having an iron core is arranged on one side of the microfluidic chamber 10 away from the permanent magnet 12. The coil itself generates a magnetic field which is indicated by the magnetic field lines 43. If this magnetic field is homogeneous over the length of the microfluidic chamber 10 and is markedly stronger than that of the permanent magnet 12, the labeling particles 11 are evenly attracted to the corresponding wall of the microfluidic chamber 10, even when the microfluidic chamber 10 in this example is arranged markedly apart from the center 44 of the permanent magnet 12. The energy required of the coil is dependent on the distance thereof from the magnet, since the magnetic force decreases with the distance. By adapting the distance to the magnet, energy can be saved, and the coil can also be supplied, for example, with smaller energy cells.

### Exemplary Embodiment 3

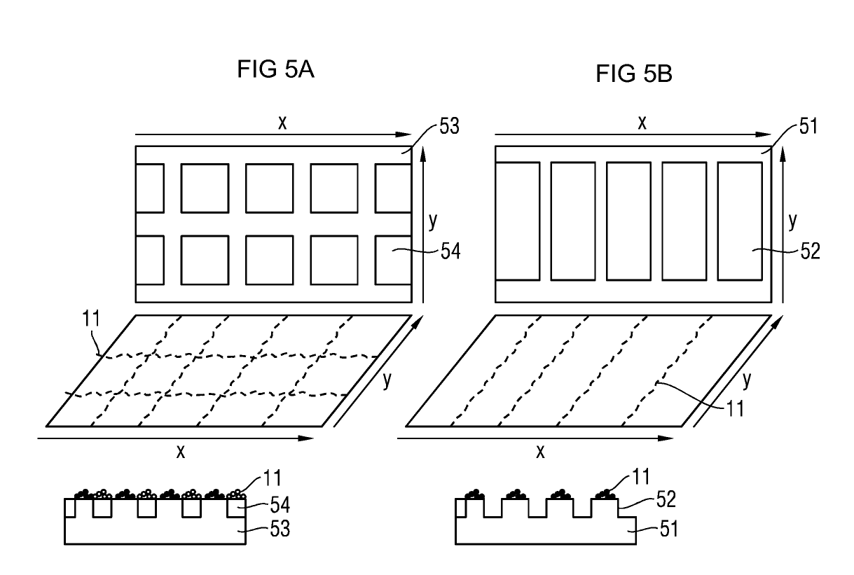

FIG. 5 shows embodiments for the iron core: In a first embodiment 51, the iron core has indentations 52, wherein the lands remaining between the indentations 52 form lines transversely to the direction of flow in the microfluidic chamber 10. The labeling particles 11 are attracted to the remaining lands and thus form lines on the surface of the microfluidic chamber 10.

In a second embodiment 53 of the iron core, the iron core has diminished indentations 54, wherein the remaining lands, in this embodiment 53, form a square line pattern. The labeling particles 11 therefore are deposited in a square pattern on the surface of the microfluidic chamber 10.

### Exemplary Embodiment 4

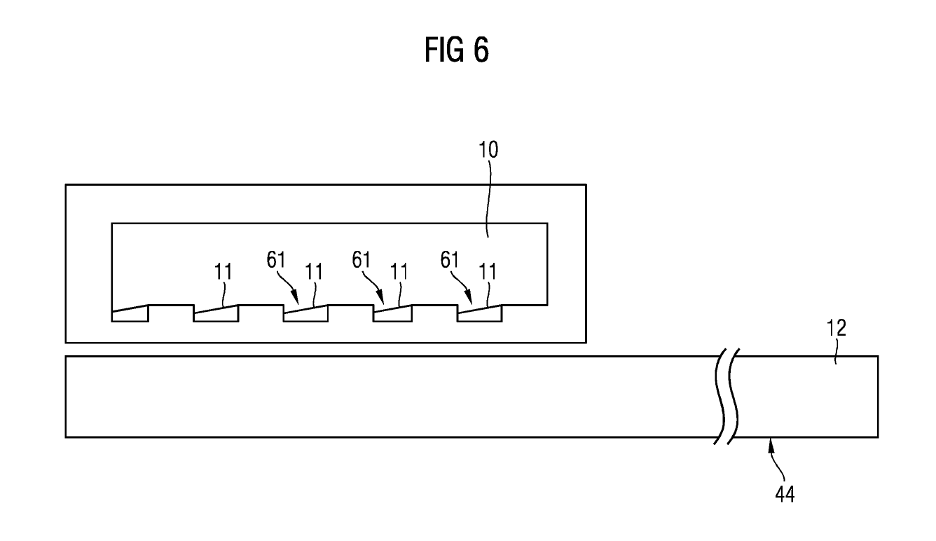

FIG. 6 shows an alternative embodiment of the microfluidic chamber 10. In this case, no coil 41 is used. Here, instead, the surface of the microfluidic chamber 10 is structured. The particles are attracted toward the center 44 of the permanent magnet 12, but, on account of the surface structuring, cannot move along the chamber surface. The particles are retained thereby at defined positions. The inner surface of the microfluidic chamber 10 on which the labeling particles 11 are deposited has notches 61 therefor. The notches 61 are uniformly distributed over the length of the microfluidic chamber 10.

The invention has been described in detail with particular reference to preferred embodiments thereof and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

What is claimed is:

1. A method for labeling cells of a cell suspension, comprising:
providing a microfluidic chamber with superparamagnetic labeling particles concentrated at substantially one inner surface of the microfluidic chamber,
charging the cell suspension into the microfluidic chamber.

2. The method as claimed in claim 1, wherein the microfluidic chamber has a cuboidal cavity with a thickness between 10 μm and 1000 μm, measured perpendicular to the inner surface on which the labeling particles are concentrated.

3. The method as claimed in claim 2, wherein said providing the microfluidic chamber comprises:
charging the microfluidic chamber with an initial suspension having the labeling particles,
concentrating the labeling particles on the inner surface of the microfluidic chamber using a magnet.

4. The method as claimed in claim 3, further comprising removing the initial suspension, excluding the labeling particles concentrated at the one inner surface of the microfluidic chamber, from the microfluidic chamber, while charging the microfluidic chamber with the cell suspension in a single pumping operation.

5. The method as claimed in claim 4, wherein the cell suspension is conducted through the microfluidic chamber in such a manner that a laminar flow is substantially present in the microfluidic chamber.

6. The method as claimed in claim 4, further comprising, after the cell suspension is charged into the microfluidic chamber and after a resting time, conducting the cell suspension to a measuring appliance.

7. The method as claimed in claim 6, wherein the resting time is less than one second.

8. The method as claimed in claim 7, further comprising counting the labeled cells in the measuring appliance.

9. The method as claimed in claim 1, wherein said providing the microfluidic chamber comprises:
charging the microfluidic chamber with an initial suspension having the labeling particles,
concentrating the labeling particles on the inner surface of the microfluidic chamber using a magnet.

10. The method as claimed in claim 9, further comprising removing the initial suspension, excluding the labeling particles concentrated at the one inner surface of the microfluidic chamber, from the microfluidic chamber, while charging the microfluidic chamber with the cell suspension in a single pumping operation.

11. The method as claimed in claim 2, further comprising removing the initial suspension, excluding the labeling particles concentrated at the one inner surface of the microfluidic chamber, from the microfluidic chamber, while charging the microfluidic chamber with the cell suspension in a single pumping operation.

12. The method as claimed in claim 1, further comprising removing the initial suspension, excluding the labeling particles concentrated at the one inner surface of the microfluidic chamber, from the microfluidic chamber, while charging the microfluidic chamber with the cell suspension in a single pumping operation.

13. An arrangement for labeling cells of a cell suspension, comprising
a microfluidic chamber having a magnet concentrating labeling particles on an inner surface of the microfluidic chamber; and
means for charging the microfluidic chamber with the cell suspension in a liquid.

14. The arrangement as claimed in claim 13, wherein the magnet is a permanent magnet arranged in a region of the microfluidic chamber.

15. The arrangement as claimed in claim 14, wherein the magnet includes a coil with an iron core arranged outside the microfluidic chamber adjacent the inner surface of the microfluidic chamber where the labeling particles are concentrated.

16. The arrangement as claimed in claim 14, wherein the iron core has indentations on an outer surface directed toward the inner surface of the microfluidic chamber.

17. The arrangement as claimed in claim 13, wherein the inner surface has notches.

18. The arrangement as claimed in claim 17, further comprising
a shared support supporting the permanent magnet and the microfluidic chamber; and
a measuring appliance counting labeled cells and arranged on the shared support with the permanent magnet and the microfluidic chamber.
